(19) United States
(12) Patent Application Publication
(10) Pub. No.: US 2021/0080079 A1
(43) Pub. Date: Mar. 18, 2021

(54) ILLUMINATION SYSTEMS AND METHOD OF USE

(71) Applicant: GENTEX CORPORATION, Zeeland, MI (US)

(72) Inventor: Xiaoxu Niu, Grand Rapids, MI (US)

(73) Assignee: GENTEX CORPORATION, Zeeland, MI (US)

(21) Appl. No.: 17/023,459

(22) Filed: Sep. 17, 2020

Related U.S. Application Data
(60) Provisional application No. 62/901,841, filed on Sep. 18, 2019.

(51) Int. Cl. F21V 9/14

(52) U.S. Cl. CPC F21V 9/14

(57) ABSTRACT

An illumination system and method of use that includes a light source, a first linear polarizer, a first converter configured to convert at least a portion of the light transmitted through the first linear polarizer to light having a first handedness of circular polarization, and a second converter configured to convert light having a second handedness of circular polarization to light having a second linear polarization. A second linear polarizer is positioned to receive the light of the second linear polarization from the second converter. At least one of the first linear polarizer and the second linear polarizer are selectively switchable between (i) a first condition in which light having the first linear polarization and light having the second linear polarization is transmitted and (ii) a second condition in which light of the first linear polarization is absorbed and light of the second linear polarization is transmitted.

### CROSS-REFERENCE TO RELATED APPLICATIONS

This application claims priority under 35 U.S.C. § 119(e) to U.S. provisional Application No. 62/901,841 filed on Sep. 18, 2019, entitled “ILLUMINATION SYSTEMS AND METHOD OF USE,” the disclosure of which is hereby incorporated by reference in its entirety.

### FIELD OF DISCLOSURE

The present disclosure generally relates to illumination systems and, more particularly, to illumination systems for use in medical procedures.

### BACKGROUND

Illumination can produce glare that is undesirable in a variety of circumstances. Glare may affect the ability of an observer to perceive details of an illuminated object, particularly in environments in which the illuminated object includes wet surfaces. Generally, the reflection of light impinging on a surface at a high angle (e.g., greater than 45 degrees relative to normal), may be linearly polarized and this reflected light may be perceived as glare by an observer. The perceived glare caused by light having a high incident angle may often be removed with an orientated absorbing or reflecting linear polarizer. However, the reflection from light impinging on a surface at angles closer to normal (e.g., less than 45 degrees relative to normal), is not polarized, and thus methods using a linear polarizer to remove glare caused by high angle light may not be effective in removing glare caused by lower angle light.

Specular reflection refers to the mirror-like reflection of light rays from a surface at the same angle to a normal vector of the surface as the incident ray, but on the opposite side of the normal vector. The reflection of light from the surface may be perceived by an observer as glare. Specular reflection may result from illumination of a smooth surface, such as a film of water. Thus, objects where moisture is present may be particularly susceptible to glare. The perceived glare as a result of specular reflection may undesirably obscure details of the object and surrounding objects. For example, during a surgical procedure, specular reflection from moisture present within the patient's body may make it difficult for a surgeon to clearly distinguish features underneath the layer of moisture and in the surrounding environment.

### SUMMARY

According to an aspect of the present disclosure, an illumination system includes a light source, a first linear polarizer, a first converter configured to convert at least a portion of the light transmitted through the first linear polarizer to light having a first handedness of circular polarization, and a second converter configured to convert light having a second handedness of circular polarization to light having a second linear polarization. A second linear polarizer is positioned to receive the light of the second linear polarization from the second converter. At least one of the first linear polarizer and the second linear polarizer are selectively switchable between (i) a first condition in which light having the first linear polarization and light having the second linear polarization is transmitted and (ii) a second condition in which light of the first linear polarization is absorbed and light of the second linear polarization is transmitted.

According to another aspect of the present disclosure, an illumination system includes a light source emitting light having a first linear polarization and a second linear polarization. A first linear polarizer is configured to absorb light of the first linear polarization and transmit light of the second linear polarization. A first converter is configured to convert the light of the second linear polarization transmitted through the first linear polarizer to light having a first handedness of circular polarization. A second converter is configured to convert light having a second handedness of circular polarization to light having the second linear polarization, wherein the light of the second handedness of circular polarization is generated from specular reflectance of the light of the first handedness of circular polarization. A second linear polarizer is configured to transmit light of the second linear polarization and selectively absorb or transmit light of the first linear polarization. The second linear polarizer is selectively switchable between (i) a first condition in which light having the first linear polarization and light having the second linear polarization is transmitted and (ii) a second condition in which light of the first linear polarization is absorbed and light of the second linear polarization is transmitted.

According to another aspect of the present disclosure, an illumination system for selectively reducing glare includes a light source emitting light having a first linear polarization and a second linear polarization. A first linear polarizer is configured to transmit light of the second linear polarization and selectively absorb or transmit light of the first linear polarization. A first converter is configured to convert the light of the second linear polarization transmitted through the first linear polarizer to light having a first handedness of circular polarization. A second converter is configured to convert light having a second handedness of circular polarization to light having the second linear polarization, wherein the light of the second handedness of circular polarization is generated from specular reflectance of the light of the first handedness of circular polarization. A second linear polarizer is configured to transmit light of the second linear polarization and absorb light of the first linear polarization. The first linear polarizer is selectively switchable between (i) a first condition in which light having the first linear polarization and light having the second linear polarization is transmitted and (ii) a second condition in which light of the first linear polarization is absorbed and light of the second linear polarization is transmitted.

According to yet another aspect of the present disclosure, a method of illuminating an object to selectively reduce glare is provided. The method includes emitting light having a first linear polarization and a second linear polarization. At least light of the second linear polarization is transmitted to a first converter. The transmitted light of the second linear polarization is converted at the first converter to light having a first handedness of circular polarization. Light is received having a second handedness of circular polarization generated from specular reflectance of the light of the first handedness of circular polarization. The light of the second handedness of circular polarization is converted by a second converter to light having the first linear polarization. At least light having the second linear polarization is provided as output.

These and other aspects, objects, and features of the present disclosure will be understood and appreciated by those skilled in the art upon studying the following specification, claims, and appended drawings.

### BRIEF DESCRIPTION OF THE DRAWINGS

In the drawings:

FIG. 1 is a schematic view of an illumination system according to aspects of the present disclosure;

FIG. 2A is a schematic view of an illumination system in a high intensity illumination configuration according to aspects of the present disclosure;

FIG. 2B is a schematic view of the illumination system of FIG. 2A in a reduced glare configuration according to aspects of the present disclosure;

FIG. 3A is a schematic view of an illumination system in a high intensity illumination configuration according to aspects of the present disclosure;

FIG. 3B is a schematic view of the illumination system of FIG. 3B in a reduced glare configuration according to aspects of the present disclosure;

FIG. 4A is a schematic view of an illumination system in a high intensity illumination configuration according to aspects of the present disclosure;

FIG. 4B is a schematic view of the illumination system of FIG. 4A in a reduced glare configuration according to aspects of the present disclosure; and

FIG. 5 is a flowchart of a method of using an illumination system according to aspects of the present disclosure.

### DETAILED DESCRIPTION

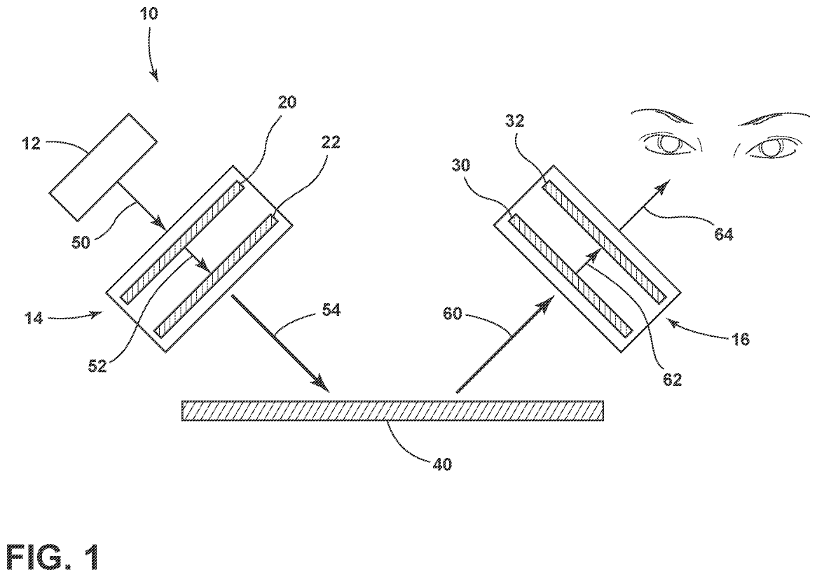

For purposes of description herein, the terms “upper,” “lower,” “right,” “left,” “rear,” “front,” “vertical,” “horizontal,” and derivatives thereof shall relate to the concepts as oriented in FIG. 1. However, it is to be understood that the concepts may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The present illustrated embodiments reside primarily in combinations of apparatus components and method steps relating to illumination systems in which it is desired to remove or reduce specular reflectance, and more particularly to illumination systems for use in medical procedures. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

Referring now to FIGS. 1-4B, reference number 10 generally designates an illumination system 10 according to an aspect of the present disclosure. The illumination system 10 includes a light source 12, an illumination component 14, and a receiving component 16. The light source 12 may be incorporated into the illumination component 14 or may be a separate component. The light source 12 can be configured to emit light having a first linear polarization and a second linear polarization. The illumination component 14 can include a first linear polarizer 20 and a first converter 22 that is configured to convert at least a portion of the light transmitted through the first linear polarizer 20 to light having a first handedness of circular polarization. The receiving component 16 can include a second converter 30 and a second linear polarizer 32. The second converter 30 can be configured to convert light having a second handedness of circular polarization to light having the second linear polarization. The second linear polarizer 32 can be positioned to receive the light of the second linear polarization from the second converter 30. At least one of the first linear polarizer 20 and the second linear polarizer 32 are selectively switchable between (i) a first condition in which light having the first linear polarization and light having the second linear polarization is transmitted and (ii) a second condition in which light of the first linear polarization is not transmitted and light of the second linear polarization is transmitted.

While the illumination component 14 is illustrated as including the first linear polarizer 20 and the first converter 22, it will be understood that these components may be housed separately or together and may optionally be incorporated into other components and/or combined with other components, such as components configured to control, support, protect, image, and/or record the illumination, for example. While the receiving component 16 is illustrated as including the second linear polarizer 32 and the second converter 30, it will be understood that these components may be housed separately or together and may optionally be incorporated into other components and/or combined with other components, such as components configured to control, support, protect, image, and/or record the illumination, for example.

Aspects of the present disclosure relate to an illumination system that can be selectively switched between a reduced glare condition and a higher illumination intensity condition. Glare is typically caused by specular reflection of illumination from a smooth surface, an example of which includes a surface of an object below a layer of liquid or moisture. This glare can decrease visibility and/or clarity of features of the object and may obscure surrounding objects and surfaces due to the brightness of the glare compared to surrounding objects and surfaces. An example scenario in which this type of glare can be challenging includes medical procedures, such as surgery, performed on humans or animals. The human body, including the tissues forming the human body, include a variety of bodily fluids that create a wet environment, non-limiting examples of which include water, blood, and sweat. These bodily fluids may be present within, around, and/or over tissues, organs, and other body components. During medical procedures, such as surgery, for example, it is often desirable to illuminate portions of the body to allow the surgeon to clearly view desired areas of the body. However, the wet environment of the human body can produce an undesirable glare due to specular reflectance of the light illuminating the body. Aspects of the present disclosure provide an illumination system and method of use that can selectively reduce glare due to specular reflectance as well as allow the illumination system to operate at a higher illumination intensity when higher power illumination is desired. The illumination system and methods of the present disclosure can be particularly useful in medical procedures that include human, animal, plant, and/or other tissues in which a wet environment may produce glare resulting from specular reflectance. However, the illumination system and methods of the present disclosure are not limited to medical procedures and may be utilized in other environments in which it is desired to control or reduce specular reflectance that can produce glare.

Still referring to FIG. 1, aspects of the illumination system 10 are illustrated with respect to a body 40, such as a human body undergoing a surgical procedure, for example, in which at least a portion of the body 40 can produce specular reflectance when illuminated by the light source 12. The light source 12 can include any source of illumination capable of generating non-polarized light, also referred to as random polarized light, illustrated by light arrow 50. Non-limiting examples of suitable light sources 12 include liquid crystal displays (LCD), laser diodes, light emitting diodes (LEDs), incandescent light sources, halogen light sources, and organic light emitting diodes (OLEDs).

The light 50 emitted by the light source 12 is directed to pass through the first linear polarizer 20. The first linear polarizer 20 can be any suitable polarizer configured to selectively not transmit light having a first linear polarization and transmit light having a second linear polarization. The first linear polarizer 20 can be a static polarizer in which light of the first linear polarization is always not transmitted or an active polarizer in which the first linear polarizer 20 can be selectively switched between a first condition in which light of the first linear polarization and light of the second linear polarization is transmitted and a second condition in which light of the first linear polarization is not transmitted and light of the second linear polarization is transmitted. Non-limiting examples of suitable linear polarizers include a wire grid polarizer and a liquid crystal polarizer. Suitable commercially available linear polarizers include those available from Midwest Optical Systems, Inc., such as an HTA008-R600 high temperature polarizer, those available from AFlash Photonics, and a PFSC super-high-contrast polarizer, available from Polarization.com.

Polarized light is often referred to as having a horizontal polarization and a vertical polarization, however, it is understood that the polarized light can be at angle, with the caveat that the polarized components of light are perpendicular to one another. Thus, for the purposes of the present disclosure, polarized light may be referred to as having a first linear polarization and a second linear polarization, where the first and second linear polarizations are perpendicular to one another. The first and second linear polarizations may include horizontal and vertical polarization or any other angle of polarization. Where the terms horizontal polarization and vertical polarization are used, it is understood that the terminology is used for the purposes of discussion and that aspects of the present disclosure may include polarizations of light at other angles, unless specified otherwise, without deviating from the scope of the present disclosure.

An example of an active polarizer includes a liquid crystal polarizer that includes a guest-host system including liquid crystal molecules (host) and dichroic dye molecules (guest). When the active polarizer is in the “off” condition, i.e., no electrical power is applied, the guest molecules absorb light of a first linear polarization and transmit light of a second linear polarization. When electrical power is supplied to the active polarizer, the molecules in the host-guest system are aligned such that the active polarizer transmits light of both the first linear polarization and the second linear polarization. Examples of suitable host liquid crystal molecules are commercially available from Merck, such as MAT-14-194. Examples of suitable guest dichroic dye molecules are commercially available from BASF, such as Irgaphor® black X 13. Other linear polarizers which are capable of being switched between a first condition in which light of both the first and second linear polarizations is transmitted light and a second condition in which light of a first linear polarization is not transmitted and light of a second linear polarization is transmitted, may also be used.

When the first linear polarizer 20 is configured to not transmit light of the first linear polarization, whether it is a static polarizer or an active polarizer in the second condition (e.g., “off” condition), the first linear polarizer 20 can be configured to not transmit at least about 30%, 40%, 50%, 60%, 70%, 80%, 90%, or 95% of the light of the first linear polarization. The first linear polarizer 20 can be configured to transmit light of a second linear polarization at a transmittance of at least about 30%, 40%, 50%, 60%, 70%, 80%, 90%, or 95%. When the first linear polarizer 20 is an active polarizer in the first condition (e.g., “on” condition), the first linear polarizer 20 can be configured to transmit light of a first linear polarization at a transmittance of at least about 30%, 40%, 50%, 60%, 70%, 80%, 90%, or 95% and transmit light of a second polarization at a transmittance of at least about 30%, 40%, 50%, 60%, 70%, 80%, 90%, 95%.

The light 52 that is transmitted by the first linear polarizer 20 travels to the first converter 22. The first converter 22 is configured to convert linearly polarized light to circular polarized light. In electrodynamics, circular polarization of light is a polarization state in which, at each point, the electric field of the light wave has a constant magnitude, but its direction rotates with time at a steady rate in a plane perpendicular to the direction of the wave. A circularly polarized wave can be in one of two possible states, right handedness circular polarization in which the electric field vector rotates in a right-hand sense with respect to the direction of propagation, and left handedness circular polarization in which the vector rotates in a left-hand sense. Elliptically polarized light may also be described as having a handedness in a substantially similar manner to that of the circularly polarized examples, but the electric vector varies in magnitude during rotation. The first converter 22 can be configured to convert light having a particular linear polarization (e.g., a first polarization or a second polarization) to light having a particular circular polarization (e.g., a right handedness or a left handedness of circular polarization). For example, aspects of the present disclosure include the first converter 22 configured to convert light of the second polarization (e.g., vertical polarization) transmitted through the first linear polarizer 20 to light having a left handedness of circular polarization.

The first converter 22 can be any suitable component configured to convert light of a particular linear polarization to light of a particular circular polarization. For example, the first converter 22 can be a quarter-wave retarder. Examples of commercially available quarter-wave retarders include those available under the tradename ZeonorFilm® ZM-Series, ZF-series, and ZD-series, commercially available from Zeon Specialty Materials.

The light 54 transmitted by the first converter 22 can be directed toward the body 40 for illuminating the body 40. The receiving component 16 can be configured to receive at least a portion of reflected light 60 that is reflected by the body 40. When a surface is illuminated at or near to normal angles of incidence with polarized light of one handedness (e.g., circular or elliptical), specular reflection of the polarized light results in conversion of the reflected light to the other handedness (e.g., left handedness to right handedness or right handedness to left handedness). The specularly reflected light may be perceived as glare to an observer.

The receiving component 16 is configured such that the reflected light 60 first interacts with the second converter 30. The second converter 30 is configured to convert circularly polarized light to linearly polarized light. The second converter 30 can be configured to convert light having a particular circular polarization (e.g., a right handedness or left handedness of circular polarization) to light having a particular linear polarization (e.g., a first polarization or a second polarization). For example, aspects of the present disclosure include the second converter 30 configured to convert light having a right handedness to light having a first linear polarization (e.g., horizontal linear polarization). The second converter 30 can be any suitable component configured to convert light of a particular circular polarization to light of a particular linear polarization. For example, the second converter 30 can be a quarter-wave retarder. Examples of commercially available quarter-wave retarders include those available under the tradename ZeonorFilm® ZM-Series, ZF-series, and ZD-series, commercially available from Zeon Specialty Materials.

The light 62 transmitted by the second converter 30 is directed toward the second linear polarizer 32. The second linear polarizer 32 can be a static polarizer in which light of a first linear polarization is not transmitted and light of a second linear polarization is transmitted or an active polarizer in which the second linear polarizer 32 can be selectively switched between a first condition in which light of a first linear polarization and light of a second linear polarization is transmitted and a second condition in which light of a first linear polarization is not transmitted and light of a second linear polarization is transmitted. Non-limiting examples of suitable linear polarizers include a wire grid polarizer and a liquid crystal polarizer. Suitable commercially available linear polarizers include those available from Midwest Optical Systems, Inc., such as an HTA008-R600 high temperature polarizer, those available from AFlash Photonics, and a PFSC super-high-contrast polarizer, available from Polarization.com. The second linear polarizer 32 can be an active liquid crystal polarizer, similar to that described above with respect to the first linear polarizer 20. Other linear polarizers which are capable of being switched between a first condition in which light of both the first and second linear polarizations is transmitted and a second condition in which light of a first linear polarization is not transmitted and light of a second linear polarization is transmitted, may also be used.

When the second linear polarizer 32 is configured to not transmit light of a second linear polarization, whether it is a static polarizer or an active polarizer in the first condition (e.g., “off” condition), the second linear polarizer 32 can be configured to not transmit at least about 30%, 40%, 50%, 60%, 70%, 80%, 90%, or 95% of the light of the first linear polarization. The second linear polarizer 32 can be configured to transmit light of a second linear polarization at a transmittance of at least about 30%, 40%, 50%, 60%, 70%, 80%, 90%, or 95%. When the second linear polarizer 32 is an active polarizer in the first condition (e.g., “on” condition), the second linear polarizer 32 can be configured to transmit light of a first linear polarization at a transmittance of at least about 30%, 40%, 50%, 60%, 70%, 80%, 90%, or 95% and transmit light of a second linear polarization at a transmittance of at least about 30%, 40%, 50%, 60%, 70%, 80%, 90%, or 95%.

Light transmitted by the second linear polarizer 32 may be provided as an output 64 for viewing and/or recording either directly to an observer and/or to a secondary system for viewing and/or recording. In one example, at least the receiving component 16 (i.e., the second converter 30 and the second linear polarizer 32) may be incorporated into eyewear or headwear worn by an observer (e.g., a surgeon or other medical provider during a medical procedure). The second converter 30 and the second linear polarizer 32 can be incorporated into a lens, shield, or film, utilized with goggles, glasses, a helmet, a visor, a face shield, or any other type of eyewear or headwear, either integrally or removably. In another example, the second converter 30 and the second linear polarizer 32 can be incorporated into a lens, shield, or film that is utilized with one or more components of an imaging system for viewing and/or recording. For example, the second converter 30 and the second linear polarizer 32 can be incorporated into a lens, shield, or film that is utilized with an optical filter for an imaging device (e.g., a video imager or CCD camera), a lens of an imaging device, a viewing window, or a display screen.

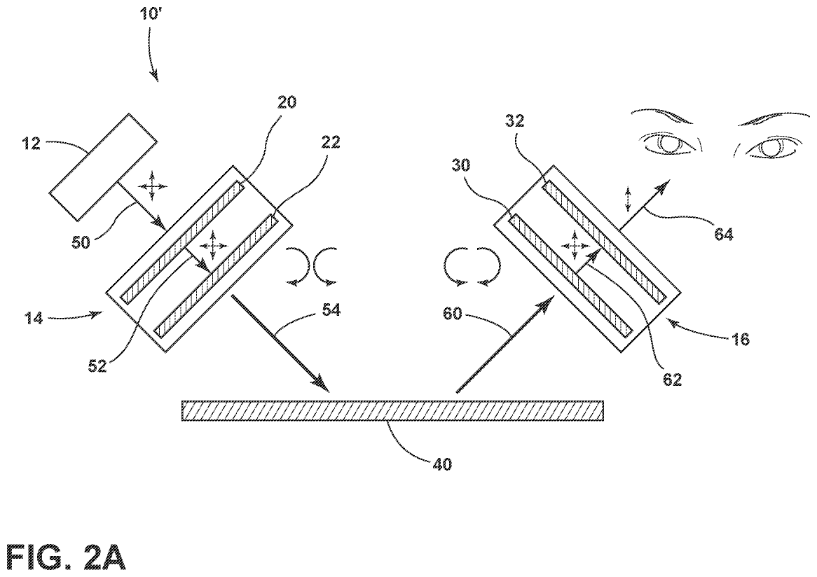

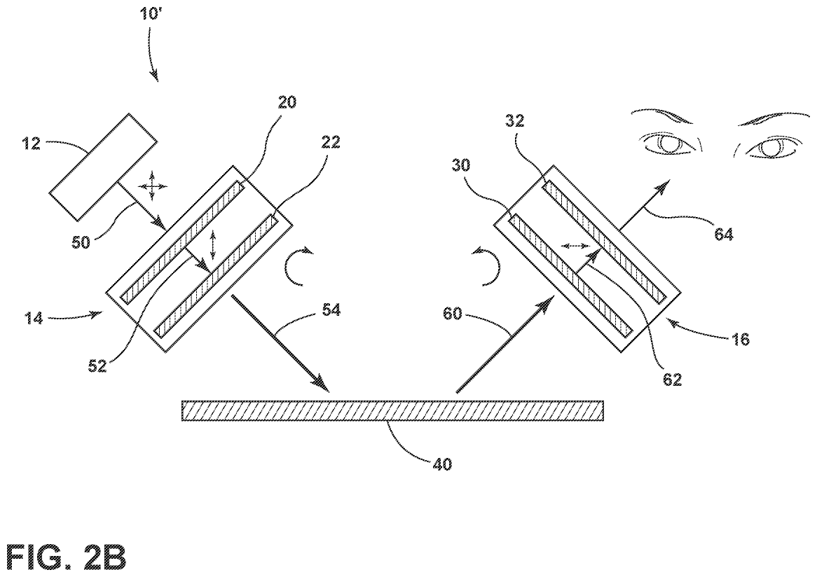

Referring now to FIGS. 2A-2B, an exemplary configuration of the illumination system 10′ according to aspects of the present disclosure. The illumination system 10′ is similar to the illumination system 10 described above with respect to FIG. 1, except that the illumination component 12 includes a first linear polarizer 20 that is an active polarizer and the receiving component 16 includes a second linear polarizer 32 that is a static polarizer. FIG. 2A illustrates the illumination system 10′ operating in a high intensity illumination condition in which the illumination system 10′ is not actively removing or filtering a portion of the light 50 emitted by the light source 12. FIG. 2B illustrates the illumination system 10′ operating in a low or reduced glare condition in which specular reflection from the body 40 is at least partially removed from the output 64 of the receiving component 16 of the illumination system 10′.

Still referring to FIGS. 2A-2B, in use, the light source 12 can be operated to emit random polarized light 50 toward the first linear polarizer 20. For the purposes of discussion, the random polarized light 50 can be considered as including a vertical polarized component and a horizontal polarized component. In the exemplary embodiment illustrated in FIGS. 2A-2B, the first linear polarizer 20 can be configured as an active polarizer that is configured to selectively switch between a first condition (FIG. 2A) in which light having a first linear polarization (horizontal polarization in the example of FIGS. 2A-2B) and light having a second linear polarization (vertical polarization in the example of FIGS. 2A-2B) is transmitted and a second condition (FIG. 2B) in which light of the first linear polarization (horizontal polarization) is not transmitted and light of the second linear polarization (vertical polarization) is transmitted. The first condition, shown in FIG. 2A, may be considered a high intensity illumination condition in that the illumination system 10′ is not actively removing or filtering a portion of the light 50 emitted by the light source 12 onto the body 40. The second condition, shown in FIG. 2B, may be considered a low or reduced glare condition in that the illumination system 10′ is being controlled to remove at least a portion of the light emitted by the light source 12 by reducing or removing specular reflection from the body 40 from the output 64 of the illumination system 10 that may be perceived as glare.

Referring now to FIG. 2A, the exemplary illumination system 10′ is illustrated in the first, high intensity illumination configuration. In the first, high intensity illumination configuration, the active, first linear polarizer 20 can be controlled to operate in the first, high intensity illumination condition in which both light having a horizontal polarization and light having a vertical polarization is transmitted through the first polarizer 20. This results in the light 54 that impinges on the body 40 having both left and right handedness of circular polarization. The light 60 reflected by the body 40 will also have both left and right handedness of circular polarization. The second converter 30 converts the circular polarized light 60 that is reflect by the body 40 to linear polarized light. Because the reflect light 60 includes both left and right handedness of circular polarization, the light 62 transmitted by the second converter 30 includes both vertically and horizontally polarized light. In the configuration of FIG. 2A, the second linear polarizer 32 is a static polarizer that is configured to always not transmit one linear polarization of light. In the configuration of FIG. 2A, the static, second linear polarizer 32 is configured to not transmit horizontally polarized light and transmit vertically polarized light as output 64. In this manner, more of the light emitted by the light source 12 is transmitted to the body 40 in the first, high intensity illumination configuration than in the second, reduced glare configuration, and thus more light is emitted in the output 64.

Referring now to FIG. 2B, the active, first linear polarizer 20 can be controlled to operate in the second, reduced glare condition such that only light having one of the linear polarizations, in this example vertical polarization, is transmitted through the first linear polarizer 20 and light of the other linear polarization, in this case horizontal polarization, is removed. In this manner, the light 52 transmitted from the first linear polarizer 20 to the first converter 22 is vertically polarized light. The first converter 22 can be configured to convert the vertically polarized light 52 into circularly polarized light, in this case light having left handedness of circular polarization. The light 54 transmitted by the first converter 22 can be directed toward the body 40 to illuminate the body 40. Because the light 54 impinging on the body 40 has a handedness of circular polarization, i.e., left handedness, specular reflection of the light 54 off the body 40 will be converted to light of the other handedness, in this case, right handedness of circular polarization. This specular reflection can be perceived as glare by an observer.

Light reflected from the body 40 can be viewed through the receiving component 16 of the illumination system 10 to reduce or remove the specularly reflect light 60 having a right handedness of circular polarization in order to reduce or remove the perceived glare. The specularly reflected light 60 having a right handedness of circular polarization can be transmitted through a second converter 30 to convert the light having a right handedness of circular polarization to light having a linear polarization. In this case, light having a right handedness of circular polarization is converted to light having a horizontal linear polarization. The horizontal polarized light 62 emitted by the second converter 30 is transmitted to the second, static linear polarizer 32 that is configured to not transmit light having a horizontal linear polarization and transmit light having a vertical linear polarization. In this manner, glare, as a result of the specularly reflected light 60, is converted to linearly polarized light 62 which can then be filtered out (reduced or removed) by the second linear polarizer 32 such that an amount of the specularly reflected light 60 in the output 64 from the receiving component 16 of the illumination system 10 is reduced or removed.

As described above with respect to FIGS. 2A-2B, in one exemplary embodiment, the first linear polarizer 20 is an active polarizer that is switchable between first and second conditions to switch between the high intensity illumination configuration and the reduced glare configuration. However, the illumination system 10 can operate in a similar manner between the high intensity illumination and the reduced glare configurations when the first linear polarizer 20 is a static polarizer that is configured to always not transmit horizontally polarized light and the second linear polarizer 32 is the active polarizer.

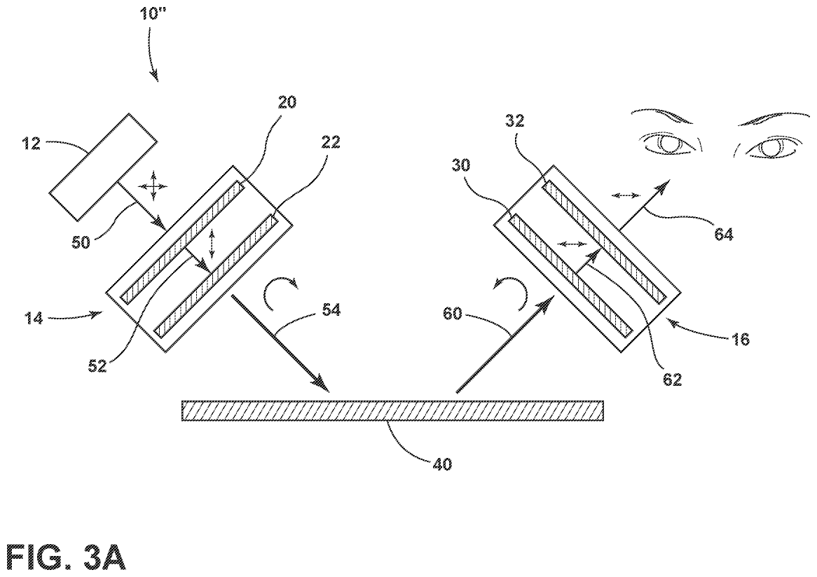

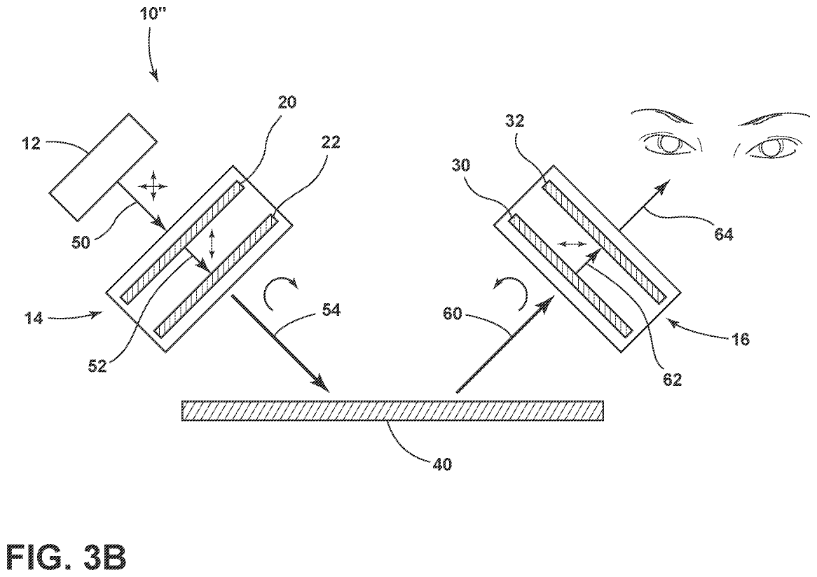

FIGS. 3A-3B illustrate an exemplary configuration of the illumination system 10″ according to aspects of the present disclosure. The illumination system 10″ is similar to the illumination system 10 described above with respect to FIG. 1, except that the illumination component 12 includes a first linear polarizer 20 that is a static polarizer and the receiving component 16 includes a second linear polarizer 32 that is an active polarizer. FIG. 3A illustrates the illumination system 10″ operating in a high intensity illumination condition in which the illumination system 10″ is not actively removing or filtering a portion of the light 50 emitted by the light source 12. FIG. 3B illustrates the illumination system 10″ operating in a low or reduced glare condition in which specular reflection from the body 40 is at least partially removed from the output 64 of the receiving component 16 of the illumination system 10″.

Referring now to FIG. 3A, the exemplary illumination system 10″ is illustrated in the first, high intensity illumination configuration. In the first, high intensity illumination configuration, the first linear polarizer 20 is a static polarizer that is always configured to not transmit one linear polarization of light and transmit another. In the configuration of FIG. 3A, the first linear polarizer 20 is configured to not transmit light having a horizontal linear polarization and transmit light 52 having a vertical linear polarization. The first converter 22 transmit the light 52 having a vertical linear polarization to the first converter 22, which is configured to convert light having a vertical linear polarization to light 54 having only a single handedness of circular polarization (left handedness of circular polarization). Specular reflection of the light 54 having only a left handedness of circular polarization by the body 40 results in the specularly reflected light 60 having the opposite handedness of circular polarization (i.e., right handedness). The specularly reflected light 60 received by the second converter 30 is converted to linearly polarized light, in this case, horizontally polarized light 62. The horizontally polarized light 62 output by the second converter 30 is transmitted to the active, second linear polarizer 32. In the high intensity illumination configuration of FIG. 3A, the active, second linear polarizer 32 can be operated in the first configuration to transmit light having both horizontal and vertical linear polarization. In the configuration of FIG. 3A, only horizontally polarized light 62 is transmitted from the second converter 30 to the second linear polarizer 32, and the horizontally polarized light 62 is transmitted by the second linear polarizer 32 as output 64.

Referring now to FIG. 3B, the exemplary illumination system 10″ is illustrated in the second, reduced glare condition. In the exemplary illumination system 10″, the first linear polarizer 20 is a static polarizer and thus the light 54 impinging on the body 40 and the specular reflectance 60 from the body 40 is the same in both the first, high intensity illumination configuration of FIG. 3B and in the second, reduced glare condition of FIG. 3B. In the second, reduced glare condition, the active, second linear polarizer 32 is operated in the unpowered condition such that vertically polarized light is transmitted and horizontally polarized light is not transmitted, such that the output 64 is free or includes a reduced amount of the specularly reflected light 60.

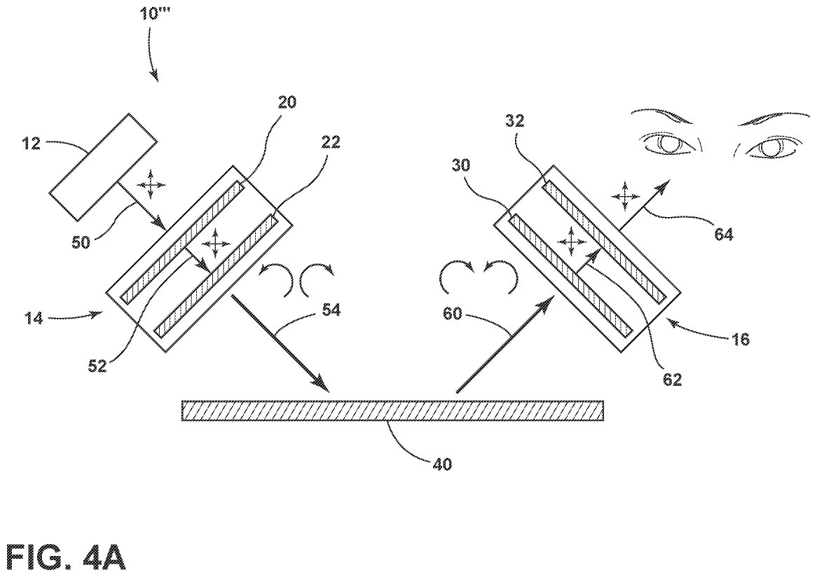

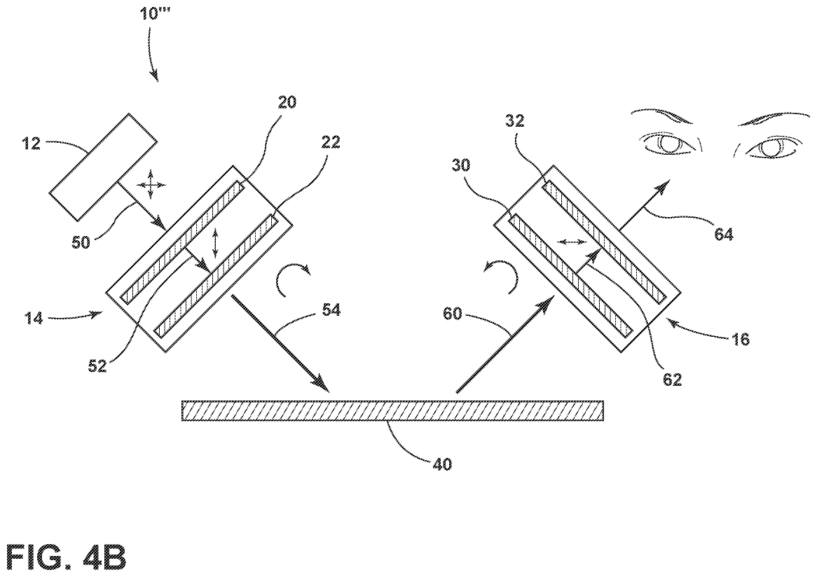

Referring now to FIGS. 4A-4B, an exemplary configuration of the illumination system 10′″ according to aspects of the present disclosure is illustrated. The illumination system 10′″ is similar to the illumination system 10 of FIG. 1, except that the illumination component 12 includes a first linear polarizer 20 that is an active polarizer and the receiving component 16 includes a second linear polarizer 32 that is also an active polarizer. FIG. 4A illustrates the illumination system 10′″ operating in a high intensity illumination condition in which the illumination system 10′″ is not actively removing or filtering a portion of the light 50 emitted by the light source 12. FIG. 4B illustrates the illumination system 10′″ operating in a low or reduced glare condition in which specular reflection from the body 40 is at least partially removed from the output 64 of the receiving component 16 of the illumination system 10′″.

Referring now to FIG. 4A, the exemplary illumination system 10′″ is illustrated in the first, high intensity illumination configuration. In this configuration, both the active, first linear polarizer 20 and the active, second linear polarizer 32 are operated in the powered on condition such that both the first and second linear polarizers 20, 32 transmit light having both vertical and horizontal polarization. In this manner, none of the light emitted by the light source 12 is filtered and any specularly reflected light 60 will be transmitted to the observer in the output 64.

Referring now to FIG. 4B, the exemplary illumination system 10′″ is illustrated in the second, reduced glare condition. In this configuration, both the active first linear polarizer 20 and the active, second linear polarizer 32 are operated in the unpowered condition such that the linear polarizers 20, 32 do not transmit light having one linear polarization and transmit light of the other linear polarization. As illustrated in FIG. 4B, in the second, reduced glare condition, the active, first linear polarizer 20 is operated to not transmit horizontally polarized light and transmit vertically polarized light. The vertically polarized light 52 transmitted by the first linear polarizer 20 to the first converter 22 is converted to light having a single handedness of circular polarization, in this case light 54 having a left handedness of circular polarization. Specular reflection of the light 54 by the body 40 results in the specularly reflected light 60 having the opposite handedness of circular polarization (right handedness of circular polarization). The specularly reflected light 60 received by the second converter 30 is converted to light having a single linear polarization, in this case horizontally polarized light 62. In the configuration of FIG. 4A, the active, second linear polarizer 32 is operated in the unpowered condition such that horizontally polarized light is not transmitted and only vertically polarized light is transmitted by the second linear polarizer 32 and provided as output 64. In this manner, glare as a result of the specularly reflected light 60 is filtered or removed from the output 64 of the illumination system 10′″.

According to one aspect of the present disclosure, the illumination system 10 can be configured such that both the first and second linear polarizers 20, 32 are static polarizers configured to operate as shown in FIG. 4B. In such a configuration, the illumination system 10 would be configured to operate in only the reduced glare condition.

While aspects of the present disclosure are discussed in the context of full or 100% efficiency of the transmittance, non-transmittance, absorbance, and conversion of the described system components, it will be understood that the components of the illumination systems described herein may operate at efficiencies less than 100% by design and/or due to natural variations, tolerances, and/or errors in the parts and materials forming the system components. It may be said that the aspects are substantially efficient.

According to one aspect of the present disclosure, an intensity of the light in the first, high intensity illumination condition and/or an amount of reduction of glare in the second, reduced glare condition can be controlled through adjustment of the active linear polarizer(s) in the illumination system 10. For example, a drive voltage and/or drive frequency of the active linear polarizer(s) can be adjusted to control the amount of absorption of light of one of the linear polarizations. For example, with reference to the exemplary configuration of FIGS. 2A-2B, the drive voltage and/or drive frequency applied to the active, first linear polarizer 20 can be controlled to adjust an amount of absorption of the horizontally polarized portion of the light 50 emitted by the light source 12.

As discussed herein, either or both of the first and second linear polarizers 20 and 32 can be active polarizers, with one of the first and second linear polarizers 20, 32 being a static polarizer. An active polarizer may require electrical power in order to be switchable between the first and second conditions, and thus may require a battery or a connection to mains electricity or other power source. When the illumination system 10 is configured to include the receiving component 16 as part of a wearable device, such as eye wear or headwear, it may be undesirable for a user of the device to carry a battery or connect to a power source, and thus it may be more desirable to have the first linear polarizer 20 be the active polarizer and use a static polarizer for the second linear polarizer 32. In other applications, it may be more desirable that the first linear polarizer 20 is a static polarizer that does not require a power source, such as may be desired for maneuverability or compactness of the illumination component 14, for example.

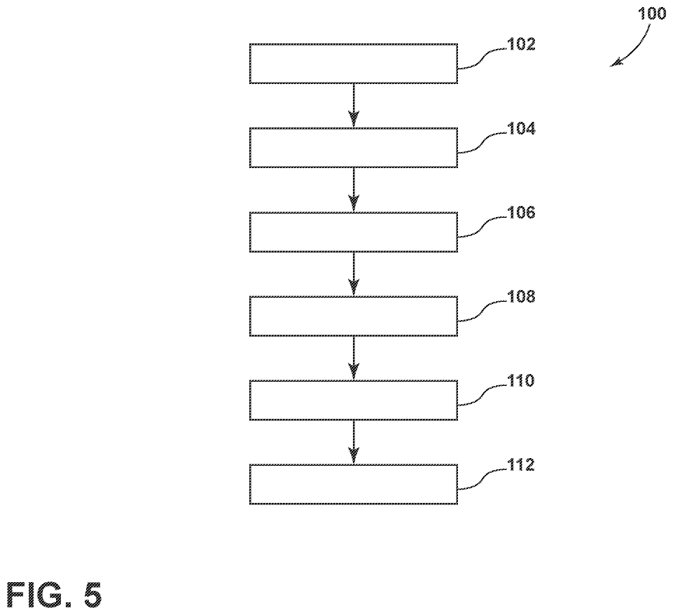

Referring now to FIG. 5, a method 100 for illuminating an object to selectively reduce glare as a result of specular reflectance according to aspects of the present disclosure is illustrated. The method 100 may be implemented using any of the illumination systems 10 disclosed herein. The method 100 may begin at 102 with emitting light having a first linear polarization and a second linear polarization from the light source 12. At 104, at least light having the second linear polarization may be transmitted to a first converter 22. The transmitting step 104 may be implemented using a static polarizer 20 that is configured to not transmit light of the first linear polarization and transmit light of a second linear polarization. Alternatively, the transmitting step 104 may be implemented using an active polarizer 20 that is selectively switchable between a first condition in which light of the first and second linear polarizations is transmitted and a second condition in which light of the first linear polarization is not transmitted and light of the second linear polarization is transmitted.

At step 106, the first converter 22 can convert the transmitted light of the second linear polarization to light having a first handedness of circular polarization. Light having the first handedness of circular polarization can be directed toward an object or surface for illumination, such as the body 40. Specular reflectance of the light having a first handedness of circular polarization produces reflected light having a second handedness of circular polarization. At step 108, light having a second handedness of circular polarization can be received by the illumination system 10. At step 110, the light having a second handedness of circular polarization can be converted to light having a first linear polarization by a second converter 30. At step 112 at least light having the second linear polarization is output from the illumination system 10. The output step 112 may be implemented using a static polarizer 32 that is configured to not transmit light of the first linear polarization and transmit light of a second linear polarization. Alternatively, the output step 112 may be implemented using an active polarizer 32 that is selectively switchable between a first condition in which light of the first and second linear polarizations is transmitted and a second condition in which light of the first linear polarization is not transmitted and light of the second linear polarization is transmitted.

In one configuration, the method 100 includes the use of an active polarizer 20 at the transmitting step 104 that can be selectively switched between (i) a first condition in which light having the first linear polarization and light having the second linear polarization is transmitted, which may be referred to as a high intensity illumination configuration, and (ii) a second condition in which light of the first linear polarization is not transmitted and light of the second linear polarization is transmitted, which may be referred to as a reduced glare configuration. In this configuration, the output step 112 may be implemented using a static polarizer configured to not transmit light of the first linear polarization and transmit light of the second polarization. However, it is within the scope of the present disclosure for the output step 112 to also include an active polarizer that is operated to provide the same functionality as the static polarizer.

In another configuration, the method 100 includes the use of an active polarizer 32 at the output step 112 that can be selectively switched between (i) a first condition in which light having the first linear polarization and light having the second linear polarization is transmitted, which may be referred to as a high intensity illumination configuration, and (ii) a second condition in which light of the first linear polarization is not transmitted and light of the second linear polarization is transmitted, which may be referred to as a reduced glare configuration. In this configuration, the transmitting step 104 may be implemented using a static polarizer that is configured to not transmit light of the first linear polarization and transmit light of the second polarization. However, it is within the scope of the present disclosure for the transmitting step 104 to also include an active polarizer that is operated to provide the same functionality as the static polarizer.

The following non-limiting aspects are encompassed by the present disclosure:

According to a first aspect of the present disclosure, an illumination system, includes: a light source emitting light having a first linear polarization and a second linear polarization; a first linear polarizer; a first converter configured to convert at least a portion of the light transmitted through the first linear polarizer to light having a first handedness of circular polarization; a second converter configured to convert light having a second handedness of circular polarization to light having the first linear polarization; and a second linear polarizer positioned to receive light transmitted by the second converter, wherein at least one of the first linear polarizer and the second linear polarizer are selectively switchable between (i) a first condition in which light having the first linear polarization and light having the second linear polarization is transmitted and (ii) a second condition in which light of the first linear polarization is not transmitted and light of the second linear polarization is transmitted.

According to a second aspect of the present disclosure, the illumination system of the first aspect, wherein the first linear polarizer is selectively switchable between (i) the first condition and (ii) the second condition, and the second linear polarizer is a static polarizer configured to not transmit light of the first linear polarization and transmit light of the second polarization.

According to a third aspect of the present disclosure, the illumination system of the first aspect, wherein the second linear polarizer is selectively switchable between (i) the first condition and (ii) the second condition, and the first linear polarizer is a static polarizer configured to not transmit light of the first linear polarization and transmit light of the second polarization.

According to a fourth aspect of the present disclosure, the illumination system of any one of the first aspect to the third aspect, wherein at least one of the first linear polarizer and the second linear polarizer includes a liquid crystal polarizer configured to switch between the first condition and the second condition.

According to a fifth aspect of the present disclosure, the illumination system of the fourth aspect, wherein the liquid crystal polarizer includes host liquid crystal molecules and guest dichroic dye molecules.

According to a sixth aspect of the present disclosure, the illumination system of any one of the first aspect to the fifth aspect, wherein at least one of the first converter and the second converter includes a quarter-wave retarder.

According to a seventh aspect of the present disclosure, the illumination system of any one of the first aspect to the sixth aspect, wherein the second converter and the second linear polarizer are incorporated into at least one of eyewear, goggles, glasses, headwear, a helmet, a visor, a face shield, a viewing window, glasses, an optical filter for an imaging device, a lens of an imaging device, and a display screen.

According to an eighth aspect of the present disclosure, an illumination system includes: a light source emitting light having a first linear polarization and a second linear polarization; a first linear polarizer configured to not transmit light of the first linear polarization and transmit light of the second linear polarization; a first converter configured to convert the light of the second linear polarization transmitted through the first linear polarizer to light having a first handedness of circular polarization; a second converter configured to convert light having a second handedness of circular polarization to light having the first linear polarization, wherein the light of the second handedness of circular polarization is generated from specular reflectance of the light of the first handedness of circular polarization; and a second linear polarizer configured to transmit light of the second linear polarization and selectively transmit light of the first linear polarization in a first condition or not transmit light of the first linear polarization in a second condition.

According to a ninth aspect of the present disclosure, the illumination system of the eighth aspect, wherein the second linear polarizer includes a liquid crystal polarizer configured to switch between the first condition and the second condition.

According to a tenth aspect of the present disclosure, the illumination system of the ninth aspect, wherein the liquid crystal polarizer includes host liquid crystal molecules and guest dichroic dye molecules.

According to an eleventh aspect of the present disclosure, the illumination system of any one of the eighth aspect to the tenth aspect, wherein at least one of the first converter and the second converter includes a quarter-wave retarder.

According to a twelfth aspect of the present disclosure, the illumination system of any one of the eighth aspect to the eleventh aspect, wherein the second converter and the second linear polarizer are incorporated into at least one of eyewear, goggles, glasses, headwear, a helmet, a visor, a face shield, a viewing window, glasses, an optical filter for an imaging device, a lens of an imaging device, and a display screen.

According to a thirteenth aspect of the present disclosure, an illumination system for selectively reducing glare, including: a light source emitting light having a first linear polarization and a second linear polarization; a first linear polarizer configured to transmit light of the second linear polarization and selectively transmit light of the first linear polarization in a first condition and not transmit light of the first polarization in a second condition; a first converter configured to convert the light of the second linear polarization transmitted through the first linear polarizer to light having a first handedness of circular polarization; a second converter configured to convert light having a second handedness of circular polarization to light having the first linear polarization, wherein the light of the second handedness of circular polarization is generated from specular reflectance of the light of the first handedness of circular polarization; and a second linear polarizer configured to transmit light of the second linear polarization and not transmit light of the first linear polarization.

According to a fourteenth aspect of the present disclosure, the illumination system of the thirteenth aspect, wherein the second linear polarizer includes a liquid crystal polarizer configured to switch between the first condition and the second condition.

According to a fifteenth aspect of the present disclosure, the illumination system of the fourteenth aspect, wherein the liquid crystal polarizer includes host liquid crystal molecules and guest dichroic dye molecules.

According to a sixteenth aspect of the present disclosure, the illumination system of any one of the thirteenth aspect to the fifteenth aspect, wherein at least one of the first converter and the second converter includes a quarter-wave retarder.

According to a seventeenth aspect of the present disclosure, the illumination system of any one of the thirteenth aspect to the sixteenth aspect, wherein the second converter and the second linear polarizer are incorporated into at least one of eyewear, goggles, glasses, headwear, a helmet, a visor, a face shield, a viewing window, glasses, an optical filter for an imaging device, a lens of an imaging device, and a display screen.

According to an eighteenth aspect of the present disclosure, a method of illuminating an object to selectively reduce glare, the method including: emitting light having a first linear polarization and a second linear polarization; transmitting at least light of the second linear polarization to a first converter; converting the transmitted light of the second linear polarization at the first converter to light having a first handedness of circular polarization; receiving light having a second handedness of circular polarization generated from specular reflectance of the light of the first handedness of circular polarization; converting the light of the second handedness of circular polarization by a second converter to light having the first linear polarization; and outputting at least light having the second linear polarization.

According to a nineteenth aspect of the present disclosure, the method of the eighteenth aspect, wherein the transmitting at least light of the second linear polarization to a first converter further includes selectively not transmitting light of the first linear polarization.

According to a twentieth aspect of the present disclosure, the method of the eighteenth aspect, wherein the outputting at least light of the second linear polarization further includes selectively not transmitting light of the first linear polarization.

According to a twenty-first aspect of the present disclosure, the method of any one of the eighteenth aspect to the twentieth aspect, wherein the outputting includes outputting the converted light from at least one of eyewear, goggles, glasses, headwear, a helmet, a visor, a face shield, a viewing window, glasses, an optical filter for an imaging device, a lens of an imaging device, and a display screen.

Modifications of the disclosure will occur to those skilled in the art and to those who make or use the concepts disclosed herein. Therefore, it is understood that the embodiments shown in the drawings and described above are merely for illustrative purposes and not intended to limit the scope of the disclosure, which is defined by the following claims as interpreted according to the principles of patent law, including the doctrine of equivalents.

It will be understood by one having ordinary skill in the art that construction of the described concepts, and other components, is not limited to any specific material. Other exemplary embodiments of the concepts disclosed herein may be formed from a wide variety of materials, unless described otherwise herein.

For purposes of this disclosure, the term “coupled” (in all of its forms: couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature, or may be removable or releasable in nature, unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, and the nature or numeral of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes, or steps within described processes, may be combined with other disclosed processes or steps to form structures within the scope of the present disclosure. The exemplary structures and processes disclosed herein are for illustrative purposes and are not to be construed as limiting.

It is also to be understood that variations and modifications can be made on the aforementioned structures and methods without departing from the concepts of the present disclosure, and further, it is to be understood that such concepts are intended to be covered by the following claims, unless these claims, by their language, expressly state otherwise.

As used herein, the term “and/or,” when used in a list of two or more items, means that any one of the listed items can be employed by itself, or any combination of two or more of the listed items, can be employed. For example, if a composition is described as containing components A, B, and/or C, the composition can contain A alone; B alone; C alone; A and B in combination; A and C in combination; B and C in combination; or A, B, and C in combination.

In this document, relational terms, such as first and second, top and bottom, and the like, are used solely to distinguish one entity or action from another entity or action, without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

What is claimed is:

1. An illumination system, comprising:
a light source emitting light having a first linear polarization and a second linear polarization;
a first linear polarizer;
a first converter configured to convert at least a portion of light transmitted through the first linear polarizer to light having a first handed polarization;
a second converter configured to convert light having a second handed polarization to light having the first linear polarization; and
a second linear polarizer positioned to receive light transmitted by the second converter,
wherein at least one of the first linear polarizer and the second linear polarizer are selectively switchable between (i) a first condition in which light having the first linear polarization and light having the second linear polarization is transmitted and (ii) a second condition in which light of the first linear polarization is not transmitted and light of the second linear polarization is transmitted.

2. The illumination system of claim 1, wherein the first linear polarizer is selectively switchable between (i) the first condition and (ii) the second condition, and the second linear polarizer is a static polarizer configured to not transmit light of the first linear polarization and transmit light of the second polarization.

3. The illumination system of claim 1, wherein the second linear polarizer is selectively switchable between (i) the first condition and (ii) the second condition, and the first linear polarizer is a static polarizer configured to not transmit light of the first linear polarization and transmit light of the second polarization.

4. The illumination system of claim 1, wherein at least one of the first linear polarizer and the second linear polarizer comprises a liquid crystal polarizer configured to switch between the first condition and the second condition.

5. The illumination system of claim 4, wherein the liquid crystal polarizer comprises host liquid crystal molecules and guest dichroic dye molecules.

6. The illumination system of claim 1, wherein at least one of the first converter and the second converter comprises a quarter-wave retarder.

7. The illumination system of claim 1, wherein the second converter and the second linear polarizer are incorporated into at least one of eyewear, goggles, glasses, headwear, a helmet, a visor, a face shield, a viewing window, glasses, an optical filter for an imaging device, a lens of an imaging device, and a display screen.

8. An illumination system for selectively reducing glare, comprising:
a light source emitting light having a first linear polarization and a second linear polarization;
a first linear polarizer configured to transmit light of the second linear polarization and selectively transmit light of the first linear polarization in a first condition and not transmit light of the first polarization in a second condition;
a first converter configured to convert the light of the second linear polarization transmitted through the first linear polarizer to light having a first handed polarization;
a second converter configured to convert light having a second handed polarization to light having the first linear polarization, wherein the light of the second handed polarization is generated from specular reflectance of the light of the first handed polarization; and
a second linear polarizer configured to transmit light of the second linear polarization and not transmit light of the first linear polarization.

9. The illumination system of claim 8, wherein the second linear polarizer comprises a liquid crystal polarizer configured to switch between the first condition and the second condition.

10. The illumination system of claim 9, wherein the liquid crystal polarizer comprises host liquid crystal molecules and guest dichroic dye molecules.

11. The illumination system of claim 8, wherein at least one of the first converter and the second converter comprises a quarter-wave retarder.

12. The illumination system of claim 8, wherein the second converter and the second linear polarizer are incorporated into at least one of eyewear, goggles, glasses, headwear, a helmet, a visor, a face shield, a viewing window, glasses, an optical filter for an imaging device, a lens of an imaging device, and a display screen.

13. A method of illuminating an object to selectively reduce glare, the method comprising:
emitting light having a first linear polarization and a second linear polarization;
transmitting at least light of the second linear polarization to a first converter;
converting the transmitted light of the second linear polarization at the first converter to light having a first handed polarization;
receiving light having a second handed polarization generated from specular reflectance of the light of the first handed polarization;
converting the light of the second handed polarization by a second converter to light having the first linear polarization; and
outputting at least light having the second linear polarization.

14. The method of claim 13, wherein the transmitting at least light of the second linear polarization to a first converter further comprises selectively not transmitting light of the first linear polarization.

15. The method of claim 13, wherein the transmitting at least light of the second linear polarization to a first converter further comprises transmitting light of the first linear polarization.

16. The method of claim 13, wherein the outputting at least light of the second linear polarization further comprises selectively not transmitting light of the first linear polarization.

17. The method of claim 13, wherein the outputting at least light of the second linear polarization further comprises transmitting light of the first linear polarization.

18. The method of claim 13, wherein the outputting comprises outputting the converted light from at least one of eyewear, goggles, glasses, headwear, a helmet, a visor, a face shield, a viewing window, glasses, an optical filter for an imaging device, a lens of an imaging device, and a display screen.

19. The method of claim 13, wherein the light having the first handed polarization is directed onto a human body.

20. The method of claim 19, wherein the specular reflectance is reflectance off of the human body.
